(19) United States
(12) Patent Application Publication
(10) Pub. No.: US 2018/0132896 A1
(43) Pub. Date: May 17, 2018

(54) APPARATUS, SYSTEMS, AND METHODS OF FACILITATING MANIPULATION OF TISSUE

(71) Applicant: COVIDIEN LP, Mansfield, MA (US)

(72) Inventor: NIKOLAI D. BEGG, WAYLAND, MA (US)

(21) Appl. No.: 15/807,676

(22) Filed: Nov. 9, 2017

Related U.S. Application Data
(60) Provisional application No. 62/421,420, filed on Nov. 14, 2016.

(51) Int. Cl. A61B 17/42; A61B 1/32; A61B 17/29

(52) U.S. Cl. CPC A61B 17/42; A61B 2017/00858; A61B 17/2909; A61B 1/32

(57) ABSTRACT

A method includes advancing a surgical device into a vaginal opening of a subject such that the surgical device remains proximal of a cervix of the subject, transitioning the surgical device from a first state to a second state such that an outer surface of the surgical device applies force to vaginal walls of the subject in a radially outward direction to frictionally engage the vaginal walls, and moving the surgical device in a caudal direction to manipulate the cervix. Surgical devices systems include a surgical device that is transitionable between the first and second states to frictionally engage the vaginal walls. The surgical device systems further include a friction-enhancing material disposed on at least a portion of the outer surface of the surgical device. The friction-enhancing material is configured to increase frictional engagement of the at least a portion of the surgical device with the vaginal walls.

### CROSS REFERENCE TO RELATED APPLICATION

The present application claims the benefit of and priority to U.S. Provisional Application Ser. No. 62/421,420, filed on Nov. 14, 2016 the entire contents of which are incorporated herein by reference.

### BACKGROUND

In gynecological procedures, a speculum may be employed to retract the interior vaginal walls to provide direct visualization and access to the cervix. For procedures that require intrauterine access, such as hysteroscopy, an instrument is typically passed through the cervix to reach the uterine cavity. The cervix is a constricted and sometimes bent or tortuous canal, and is held in place by soft compliant tissue. Thus, insertion of the instrument may be difficult for the clinician and/or painful for the patient.

A tenaculum may be used in conjunction with the speculum to facilitate access to the uterine cavity. In use, the surgeon grasps the outer edge of the cervix with the tenaculum and applies tension away from the uterus while advancing the instrument through the cervix. By pulling the cervix towards the vaginal opening, the cervix is stabilized and straightened and the cervical canal aligned with the instrument. As a result, the instrument more easily passes through the cervix and into the uterine cavity. However, in grasping the cervix, tenaculum may damage tissue and/or cause pain to the patient.

### SUMMARY

Provided in accordance with aspects of the present disclosure is a method including advancing a surgical device into a vaginal opening of a subject to a position proximal of a cervix of the subject, transitioning the surgical device from a first state to a second state such that an outer surface of the surgical device applies force to vaginal walls of the subject in a radially outward direction relative to a longitudinal axis of the surgical device to frictionally engage the vaginal walls of the subject, and moving the surgical device in a caudal direction to manipulate the cervix of the subject.

In an aspect of the present disclosure, the surgical device is a speculum including a handle and first and second arms coupled to the handle. In such aspects, transitioning the surgical device from the first state to the second state includes moving the first and second arms from a closed position to an open position.

In another aspect of the present disclosure, the surgical device is a forceps including a handle and first and second arms coupled to the handle. In such aspects, transitioning the surgical device from the first state to the second state includes moving the first and second arms from a closed position to an open position.

In another aspect of the present disclosure, the surgical device is a collapsible mesh structure (CMS). In such aspects, transitioning the surgical device from the first state to the second state includes expanding the CMS from a radially collapsed condition to a radially expanded condition.

In still another aspect of the present disclosure, expanding the CMS from the radially collapsed condition to the radially expanded condition includes applying compressive force along a longitudinal axis of the CMS.

In yet another aspect of the present disclosure, in the radially collapsed condition, the CMS defines a first length, while, in the radially expanded condition, the CMS defines a second length less than the first length.

In still yet another aspect of the present disclosure, the surgical device includes a friction enhancing material disposed on the outer surface thereof. The friction enhancing material increases frictional engagement with the vaginal walls of the subject upon movement of the surgical device in the caudal direction to manipulate the cervix of the subject.

In another aspect of the present disclosure, the method further includes inserting a surgical instrument through the surgical device and into the cervix of the subject. The method may also include performing at least one surgical task on or within the cervix of the subject with the surgical instrument. Further, the method may include removing the surgical instrument, transitioning the surgical device from the second state back to the first state, and removing the surgical device from the vaginal opening of the subject.

In yet another aspect of the present disclosure, prior to advancing the surgical device into the vaginal opening of the subject, a friction-enhancing material is applied to the outer surface of the surgical device.

A system provided in accordance with aspects of the present disclosure includes a surgical device defining an outer surface and a longitudinal axis. The surgical device is configured for insertion into a vaginal opening of a subject and is transitionable between a first state, wherein the surgical device defines a first radial dimension, and a second state, wherein the surgical device defines a second radial dimension greater than the first radial dimension. In the second state, the outer surface of the surgical device is configured to frictionally engage vaginal walls of the subject. The system further includes a friction-enhancing material disposed on at least a portion of the outer surface of the surgical device. The friction-enhancing material is configured to increase the frictional engagement of the at least a portion of the surgical device with the vaginal walls of the subject.

In an aspect of the present disclosure, the surgical device is a speculum including a handle and first and second arms coupled to the handle. The handle is selectively manipulatable to transition the first and second arms from a closed position, corresponding to the first state, to an open position, corresponding to the second state.

In another aspect of the present disclosure, the surgical device is a forceps including a handle and first and second arms coupled to the handle. The handle is selectively manipulatable to transition the first and second arms from a closed position, corresponding to the first state, to an open position, corresponding to the second state.

In still another aspect of the present disclosure, the surgical device is a CMS configured to transition between a radially collapsed condition, corresponding to the first state, and a radially expanded condition, corresponding to the second state. In the radially collapsed condition, the CMS may define a first length and a first diameter, and, in the radially expanded condition, the CMS may define a second length less than the first length and a second diameter greater than the first diameter.

In yet another aspect of the present disclosure, the CMS includes a plurality of interwoven components.

In still yet another aspect of the present disclosure, the CMS is configured to transition from the radially collapsed condition to the radially expanded condition in response to application of a compressive force thereto.

In another aspect of the present disclosure, the system further includes a surgical instrument configured for insertion through the surgical device when the surgical device is in the second state.

In another aspect of the present disclosure, the friction-enhancing material is gauze.

### BRIEF DESCRIPTION OF THE DRAWINGS

Aspects and features of the present disclosure are described in detail below with reference to the accompanying drawings wherein:

FIG. 1 is a side view of a forceps including a friction-enhancing material disposed on a portion thereof in accordance with an embodiment of the present disclosure;

FIG. 2 is a side view of a speculum including a friction-enhancing material disposed on a portion thereof in accordance with an embodiment of the present disclosure;

FIG. 3A is a perspective view of a collapsible mesh structure including a friction-enhancing material provided in accordance with the present disclosure, wherein the collapsible mesh structure is disposed in a collapsed state;

FIG. 3A′ is a transverse, cross-sectional view of the collapsible mesh structure of FIG. 3A, wherein the collapsible mesh structure is disposed in the collapsed state;

FIG. 3B is a perspective view of the collapsible mesh structure including the friction enhancing material of FIG. 3A, wherein the collapsible mesh structure is disposed in an expanded state;

FIG. 3B′ is a transverse, cross-sectional view of the collapsible mesh structure of FIG. 3B, wherein the collapsible mesh structure is disposed in the expanded state;

FIGS. 4A and 4B illustrate use of the collapsible mesh structure of FIG. 3A, in conjunction with an instrument, during surgery;

FIG. 5 is a flow chart of a method of surgery provided in accordance with the present disclosure; and

FIG. 6 is a schematic illustration of a robotic surgical system configured for use in accordance with the present disclosure.

### DETAILED DESCRIPTION

The apparatus, systems, and methods of the present disclosure may find particular applicability for use in manipulating a subject's cervix, More specifically, the apparatus, systems, and methods of the present disclosure may be utilized to apply radial force to a subject's vaginal walls and facilitate friction with the vaginal walls (which are naturally lubricated and hydrated) to enable the transfer of tensile force to the vaginal walls, in the form of a pulling in the caudal direction (towards the vaginal opening), thus enabling pulling of the vaginal walls in the caudal direction to thereby manipulate the cervix and facilitate insertion of an instrument therethrough. In this manner, the need to directly grasp the cervix is obviated and, as a result, tissue damage and pain to the patient are minimized. As used herein with respect to tissue, “manipulate” means to affect the position or orientation of tissue by direct contact with the tissue, or indirectly by contact with adjacent tissue (e.g., contact of the surgical device with the vaginal walls will manipulate the cervix). Manipulation of tissue may move, re-position, straighten, adjust, or stabilize tissue, for example. Although detailed hereinbelow with respect to use in gynecological procedures, the apparatus, systems, and methods of the present disclosure may also find applicability in other procedures to facilitate access to and manipulation of other anatomical structures that may be accessed through a body aperture other than the vagina, such as for example, through the mouth, anus, or a surgically created aperture. In addition, it should be understood that apparatus, systems, and methods of the present disclosure may find particular applicability in the veterinary field as well as in the human medical field.

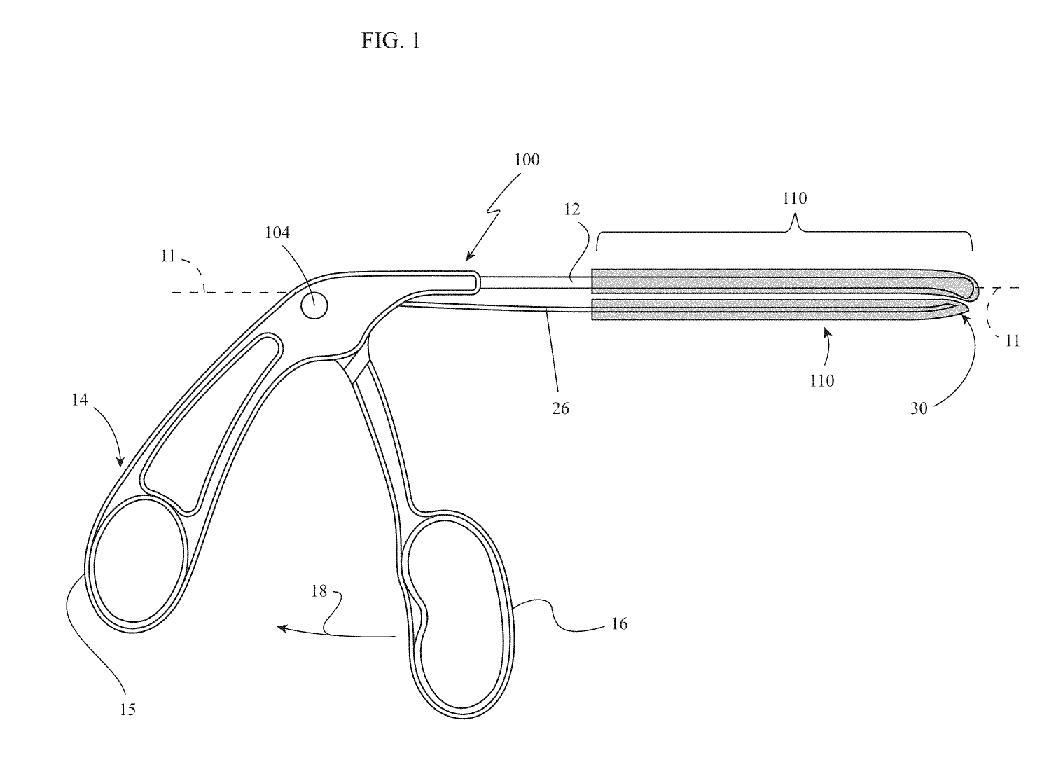

FIG. 1 shows a forceps 100 according to some embodiments of the present disclosure. The forceps 100 include a distal portion 30, a central axis 11, and a handle 14. Handle 14 includes a movable finger grip 16 and a stationary thumb grip 15 connected at a pivot point 104. Forceps 100 further includes a first arm 12 and a second arm 26 movable relative to the first arm 12 between an open position and a closed position (FIG. 1). Arrow 18 indicates a direction in which the movable finger grip 16 may be manipulated in order to move the second arm 26 relative to the first arm 12 from the closed position towards the open position.

A friction-enhancing material 110 is disposed on at least a portion of the outer surface of each of the first and second arms 12 and 26, respectively, of forceps 100. While one specific configuration of the friction-enhancing material 110 is shown in FIG. 1, one or more friction-enhancing materials 110 may be employed and disposed in various configurations along a larger or smaller portion of the first and second arms 12 and 26, respectively. By providing friction-enhancing material 110 on portions of the outer surfaces of arms 12, 26, e.g., on the surfaces configured to contact the vaginal walls in the open position during use, frictional engagement between the outer surfaces of arms 12, 26 and the vaginal walls is facilitated, thus enabling the application of tensile force to the vaginal walls to adjust and stabilize the cervix while minimizing trauma to surrounding tissue and pain to the patient.

The friction-enhancing material 110 may be any material that inhibits slippage between the surgical device, e.g., first and second arms 12, 26, respectively, of forceps 100, and tissue, such as the vaginal walls. In embodiments, a portion of the outer surface of the surgical device may be coated with the friction-enhancing material 110. In other embodiments, a pre-formed layer of friction-enhancing material 110 may be applied to at least a portion of the surgical device. In yet other embodiments, friction-enhancing material 110 may be applied to a structure that is then mounted on at least a portion of the surgical device. In embodiments, the friction-enhancing material 110 may be a textile (such as gauze) or a biocompatible coating (such as an adhesive material).

The friction-enhancing material 110 may be formed in whole or in part from a friction-enhancing composition that includes a polymer having a higher coefficient of friction than the outer surface of the surgical device and which is high enough that the outer surface of the surgical device slips less than it would without the friction-enhancing material when moved against the vaginal wall or other tissue. Coefficient of friction may be determined per ASTM D3702 against a polished steel surface. In embodiments, the friction-enhancing material 110 has a coefficient of friction value of about 0.7 or higher, in other embodiments about 0.8 and higher. Exemplary friction-enhancing materials 110 include, but are not limited to rubbery elastomeric thermoplastic polymers, such as, for example, styrene-olefin block copolymers and acrylonitrile block copolymers, urethane-based thermoplastic elastomers, ester-based thermoplastic elastomers, olefin-based thermoplastic elastomers, and/or amide-based thermoplastic elastomers. Linear low density polyethylene, very low density polyethylene, polyethylene-α-olefin copolymers or polycarbonate-urethane copolymers may also be suitable for use in the friction-enhancing composition.

In embodiments, the friction-enhancing composition includes one or more styrene-olefin thermoplastic elastomers. A styrene-olefin thermoplastic elastomer is a block copolymer having a soft segment and a hard segment within a molecule. The soft segment is a unit that is obtained from polymerization of an olefin, e.g., a polyisobutylene block, a polybutadiene block or a polyisoprene block. The component constituting the hard segment is a unit of styrene block, for example, that is obtained from a compound having one or at least two types selected from styrene and its derivatives, e.g., α-methyl styrene, vinyl toluene, p-tertiary butyl styrene, 1,1-diphenyl ethylene and others. Specific examples of the styrene-olefin thermoplastic elastomers include: styrene-isobutylene-styrene block copolymer (SIBS); styrene-butadiene-styrene block copolymer (SBS); styrene-ethylene-butylene-styrene block copolymer (SEBS); styrene-isoprene-styrene block copolymer (SIS); styrene-ethylene-propylene-styrene block copolymer (SEPS); styrene-ethylene-ethylene-propylene-styrene block copolymer (SEEPS structure); and modified block copolymers thereof. The content of styrene (or its derivatives) in each of the SIBS, SBS, SEBS, SIS, SEPS and SEEPS structures may in embodiments be in a range of 10-50 wt. %, and in embodiments in a range of 15-45 wt. % within the copolymer, in yet other embodiments SIBS with about 17 wt % styrene.

The friction-enhancing material 110 may also be a coating made from a composition that includes an adhesive that is biocompatible when set. For instance, the adhesive may be one that provides at least some tack during application to the outer surface of the surgical device. The adhesive may be a pressure sensitive, hot melt, solution, dispersion or curable material.

When the friction-enhancing material 110 is a coating, it may be applied from a solution or dispersion. In the case of a hot melt or curable adhesive, the coating may be applied neat. Suitable coating thicknesses may be, in embodiments, from about 1 to about 25 μm, in other embodiments from about 2 μm to about 20 μm, and in yet other embodiments from about 5 to about 10 μm.

In addition to, or in lieu of being made in whole or in part from a friction-enhancing composition, the friction-enhancing material 110 may include a surface texture that inhibits slippage. Thus, in embodiments, the friction-enhancing material 110 may be a textile (woven, non-woven, knitted, braided, and the like) having a surface texture provided during or after manufacture. In embodiments where the friction-enhancing material 110 is a film or coating, a surface texture may be provided during or after manufacture or application, and may include, for example, ridges, pores, recesses, protrusions, or other structure provided on the film or coating.

In embodiments, the friction-enhancing material 110 is made in whole or in part of a material that absorbs water, so that the outer surface of the surgical device slips less than it would without the friction-enhancing material 110 when moved against the vaginal wall or other tissue.

In some embodiments, the friction-enhancing material 110 is disposed on the surgical device, e.g., arms 12, 26 of forceps 100, during an initial manufacturing process at an original equipment manufacturer (OEM). In other embodiments, the friction-enhancing material 110 is reapplied by the OEM when the forceps 100 (or other surgical device) is sent for maintenance, repair, and/or reprocessing. In still other embodiments, the friction-enhancing material 110 is be initially disposed on the forceps 100 (or other surgical device) and/or may be enhanced prior to use by a practitioner (e.g., a nurse or doctor).

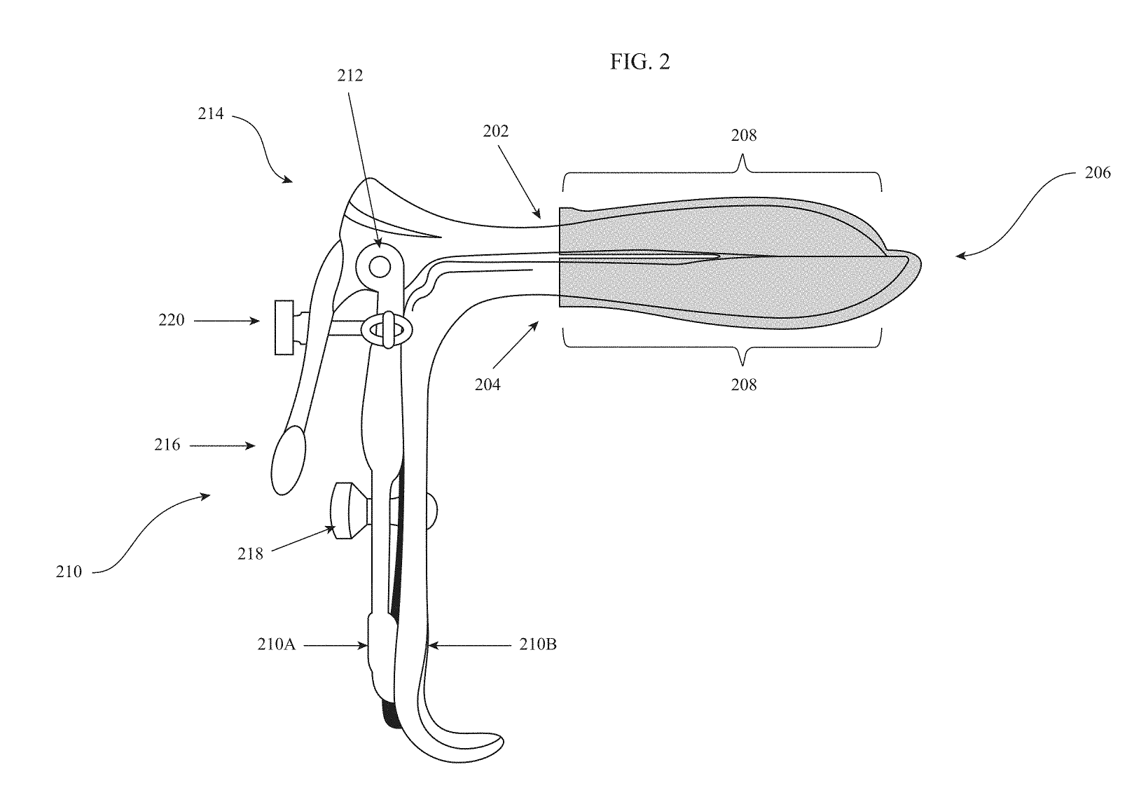

FIG. 2 shows a speculum 200 according to some embodiments of the present disclosure. Speculum 200 includes a distal portion 206, a proximal portion 214, a first arm 202, and a second arm 204. Speculum 200 further includes a handle 210 having a movable component 216 and two stationary components 210A and 210B that may be adjusted relative to one another and fixed in position via a ratchet 218 or other suitable mechanism. The movable component 216 may be depressed towards the stationary components 210A, 210B during use to move the first arm 202 relative to the second arm 204 from a closed position to an open position. A ratchet 220 or other suitable mechanism may be utilized to adjust the range of motion of the movable component 216. In some embodiments, the second arm 204 is formed integrally with or otherwise fixed relative to the second stationary component 210B, while the movable component 216 and first stationary handle 216, 210A, respectively, are coupled to the first arm 202 via at least one hinge 212.

A friction-enhancing material 208 may be disposed on a portion of the outer surface of each of the first and second arms 202, 204. As illustrated in FIG. 2, the friction-enhancing material 208 is disposed on distal portions of the arms 202, 204, extending from the distal portion 206 of the speculum 200 towards the proximal portion 214. The friction-enhancing material 208 may be similar to and/or disposed on speculum 200 similarly as friction-enhancing material 110 (FIG. 1).

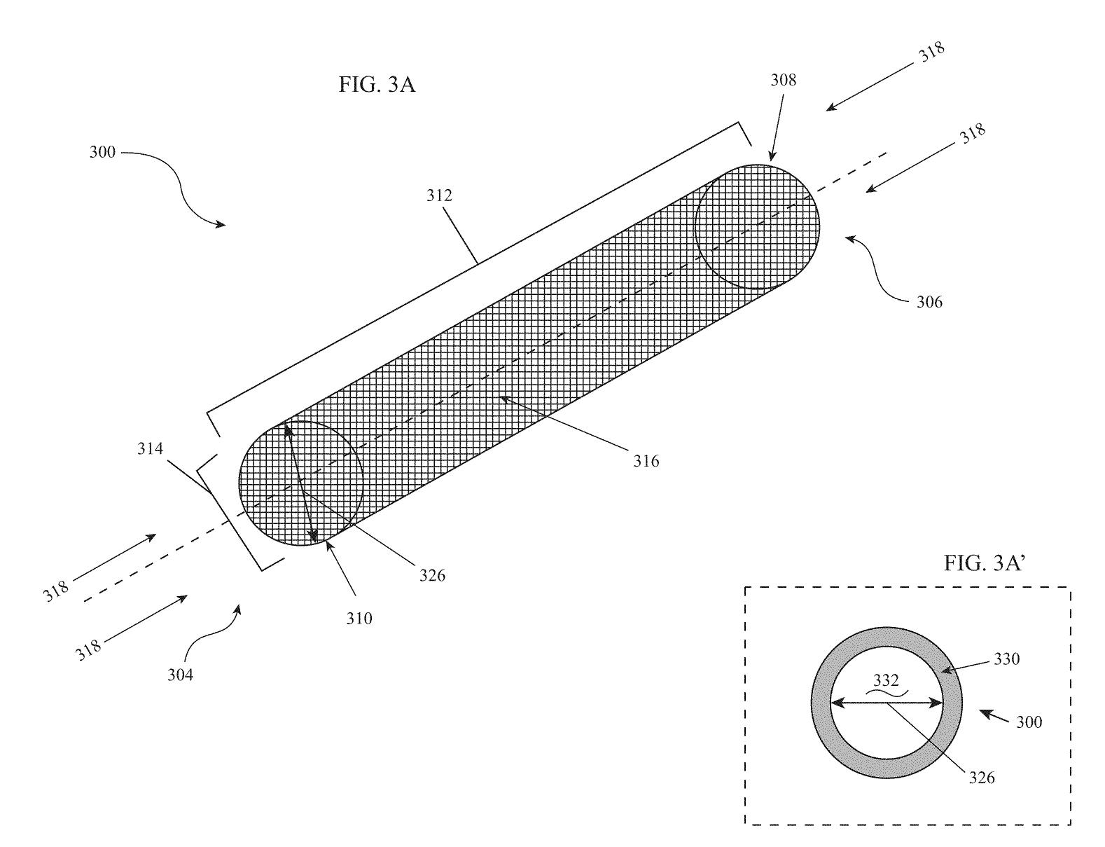

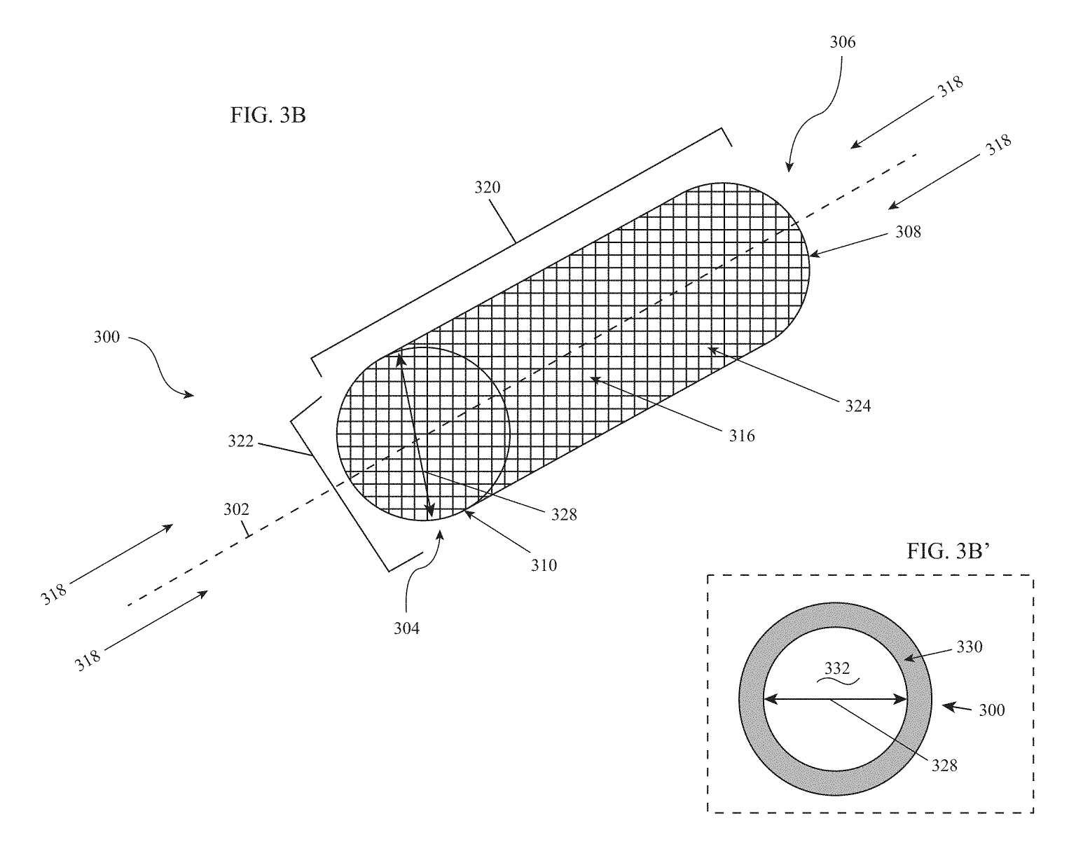

FIGS. 3A, 3A′, 3B, and 3B′ illustrate a collapsible mesh structure (“CMS”) 300 according to some embodiments of the present disclosure. The CMS 300 is configured such that axial compression of the CMS 300 results in radial expansion thereof. The CMS 300 may be constructed of cross-braided cord or strips comprised of biocompatible textile, polymer, and/or metallic materials. In some embodiments, the CMS 300 may include one or more contiguous components (threads, wires, ropes, etc.) woven together, where each component is at least the length of the CMS 300 in the expanded (FIGS. 3A and 3A′) and/or collapsed (FIGS. 3B and 3B′) condition thereof. In other embodiments, the CMS 300 may include components having sections less than the length of the CMS 300 and coupled together via a hinge, a weld, or other suitable coupling. The CMS 300 may additionally or alternatively be designed as braided wire or as woven semi-rigid polymer sleeve that exhibits “Poisson Effect” behavior, e.g., an absorbent textured material. The CMS 300, in any of the above embodiments, may be formed from friction-enhancing material 330 (e.g., absorbent textured material) and/or may be coated with a friction-enhancing material 330. For example, a CMS 300 of woven semi-rigid polymer may be further coated with a friction-enhancing material 330 on its outer surface. Thus, the outer surface of the CMS 300 is coated or covered with the friction-enhancing material 330 which serves to absorb moisture and decrease lubrication on the vaginal walls to enhance frictional engagement therewith.

FIG. 3A illustrates CMS 300 in the collapsed condition. CMS 300 includes a central axis 302, a first end portion 304 defined by a first collapsible mechanism 310, a second end portion 306 defined by a second collapsible mechanism 308, an interior diameter 326 (in the collapsed condition), an outer diameter 314 (in the collapsed condition), and a plurality of interwoven components 316. In embodiments, the first and the second collapsible mechanisms 310, 308 include collapsible/expandable rings, although other mechanisms may also be provided, such as, for example, selectively fillable bladders, electro-active elements configured to expand (or contract) in response to application of electrical energy thereto, shape-memory elements configured to expand (or contract) in response to application of thermal energy thereto, deployable scaffolding configured to expand (or contract) in response to mechanical forces (axial force, rotational force, and combinations thereof) imparted thereto, etc. The first and second collapsible mechanisms 310, 308 may be utilized to anchor at least some of the plurality of interwoven components 316 and assist in the transition between the collapsed and expanded conditions. An overall length 312 of the CMS 300 in the collapsed condition may be from about 1.0″ to about 6.0″, in embodiments from about 2.0″ to about 4.0″ and the interior diameter 326 in the collapsed condition may be from 0.25″ to about 2.0″, in embodiments, from about 0.6″ to about 1.0″.

In some embodiments, at least some of the plurality of interwoven components 316 are anchored at the first end portion 304 and/or at the second portion 306 via the first collapsible mechanism 310 and the second collapsible mechanism 308, respectively. In these and other embodiments, the first and/or the second collapsible mechanisms 310 and 308 are, as noted above, collapsible/expandable rings, for example, those defining petal-like configurations (not explicitly shown) such that the rings are configured to expand in response to a compression force 318 and retract when the compression force 318 is removed and/or an opposite force is applied. In other embodiments, the first and/or the second collapsible mechanisms 310 and 308 are formed at least in part by the plurality of interwoven components 316. At least some of the plurality of interwoven components 316 may be braided, threaded, twisted, or otherwise configured to form the first and/or second collapsible mechanisms 310, 308. In such embodiments, the least some of the plurality of interwoven components 316 are arranged to expand in response to the compression force 318 and retract when the compression force 318 is removed and/or an opposite force is applied.

In other embodiments, some of the plurality of interwoven components 316 are coupled to each other but may not be coupled to either of the first collapsible mechanism 310 or the second collapsible mechanism 308, or may be coupled to only one of the first collapsible mechanism 310 or the second collapsible mechanism 308. Each component of the plurality of components 316 may comprise a single wire, string, strand, or a plurality of strands that may be woven or twisted together, and may be interconnected directly (e.g., via soldering, hinging, etc.) or indirectly (e.g., via weaving, tying, binding, etc.).

Referring to FIG. 3A′, a cross-sectional view of the CMS 300 in the collapsed condition is shown. A channel 332 extends the length 312 (FIG. 3A) of the CMS 300. Also illustrated in FIG. 3A′ is the friction-enhancing material 330. The friction-enhancing material 330 may be disposed on the outer periphery of the CMS 300 along a portion or the entire length 312 (FIG. 3A) thereof and/or about a portion or the entire circumference of the CMS 300. Alternatively or additionally, some or all of the plurality of interwoven components 316 may be formed from a friction-enhancing material 330 such that the outer periphery of the CMS 300 exhibits friction-enhancing characteristics. The friction-enhancing material 330 may comprise varying thicknesses and materials, such as those detailed above, and may be integral with or disposed on the CMS 300 (similarly as detailed above).

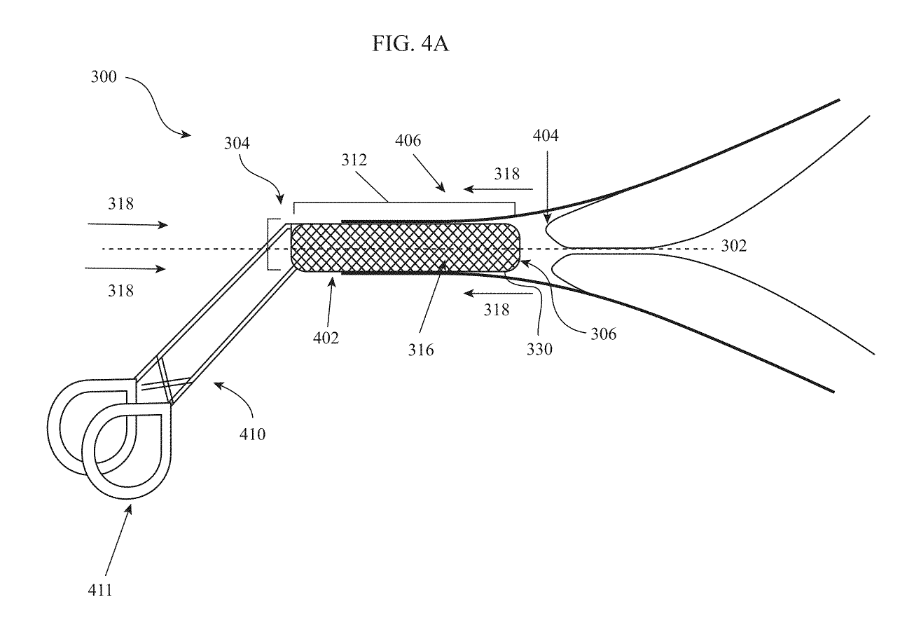

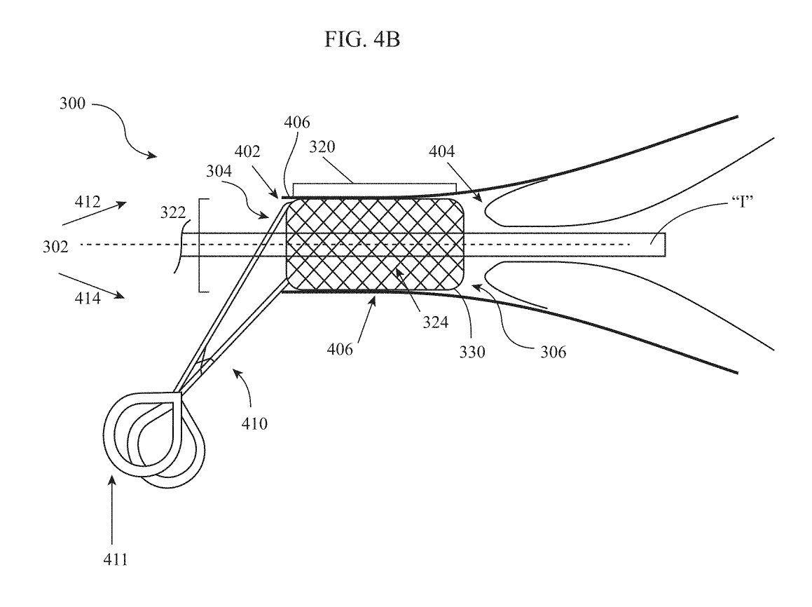

FIG. 3B illustrates the CMS 300 in the expanded condition, wherein the CMS 300 defines an outer diameter 322, an inner diameter 328, and an overall length 320. As noted above, the plurality of interwoven components 316 of the CMS 300 are configured such that, when a compression force 318 (application of inward axial force from both end portions 304, 306 or from one end portion 304, 306 with the other end portion 304, 306 maintained in fixed position) is applied along the central axis 302, e.g., by way of actuating a pair of handles 411 (FIGS. 4A and 4B), as detailed below, or other suitable compression-applying device, the CMS 300 compresses axially and expands radially, resulting in the expanded condition shown in FIG. 3B. In response to the radial expansion of the CMS 300 to the expanded condition, a plurality of spaces 324 are enlarged and/or formed among and between the plurality of interwoven components 316. In some embodiments, the overall length 320 of the CMS 300 in the expanded condition may be from about 0.5″ to about 5″, in embodiments from about 2″ to about 4″ and the interior diameter 328 may be from about 0.5″ to about 4″, in embodiments from about 1.0″ to about 3.0.″ Thus, the outer diameter 322 of the CMS 300 in the expanded condition is greater than the outer diameter 314 of the CMS 300 in the collapsed condition (FIG. 3A). The inner diameter 328 of the CMS 300 in the expanded condition is likewise greater than the inner diameter 326 of the CMS 300 in the collapsed condition (FIG. 3A). However, the overall length 320 of the CMS 300 in the expanded condition is less than the overall length 312 of the CMS in the collapsed condition (FIG. 3A).

In some embodiments, once the CMS 300 is transitioned to the expanded condition, the compression force 318 applied to cause the transition may be maintained in order to maintain the CMS 300 in the expanded condition.

FIG. 3B′ is a cross-sectional view of the CMS 300 in the expanded condition, illustrating the channel 332 of the CMS 300 and the friction-enhancing material 330. Expansion of the CMS 300 from the collapsed condition (FIGS. 3A and 3A′) to the expanded condition (FIGS. 3B and 3B′) does not compromise the integrity or the functionality of the friction-enhancing material 330.

Referring generally to FIGS. 3A-3B′, in use, the CMS 300 is inserted into the vagina in the collapsed condition (FIGS. 3A and 3A′). Once inserted, the CMS 300 is compressed in the axial direction, e.g., by applying axial force to the first collapsible mechanism 310 and/or the second collapsible mechanism 308. As the overall length of the CMS 300 decreases, the CMS 300 expands radially outwards, contacting the vaginal walls in frictional engagement therewith. The expanding action retracts the vaginal walls to provide access and visualization of the cervix. The shortening action of the CMS 300 in the axial direction pulls the vaginal walls, and subsequently the cervix, towards the vaginal opening, thus straightening and stabilizing the cervix.

In addition, just as the plurality of interwoven components 316 are configured to allow the expansion shown in FIG. 3B, the plurality of interwoven components 316 may also be configured to allow for the adjustment of the CMS 300 along or relative to the central axis 302 thereof to thereby adjust the position of the cervix.

FIGS. 4A and 4B illustrate the CMS 300 in use in connection with an instrument 410. FIG. 4A, more specifically, illustrates CMS 300 in the collapsed condition, for insertion through an opening such as vaginal opening 402. Instrument 410 may be removably coupled to the first end portion 304 of the CMS 300 at one or more locations. Instrument 410 includes a pair of handles 411, although other suitable configurations are contemplated. Handles 411 may be manipulated relative to one another to transition CMS 300 between the collapsed and expanded conditions. Each handle 411, more specifically, may be operably coupled to one of the collapsible mechanisms 310, 308 and/or one of the end portions 304, 306 of the CMS 300 such that manipulation of handles 411 relative to one another, e.g., relative pivoting, rotation, and/or translation, translates and/or rotate the collapsible mechanisms 310, 308 relative to one another to transition CMS 300 between the collapsed and expanded conditions. Handles 411 may be removably coupled to either or both of collapsible mechanisms 310, 308 (FIGS. 3A and 3B) or may replace the same. A cervix 404 is also illustrated in FIG. 4A, and it is noted that the second end portion 306 of the CMS 300, once inserted through the vaginal opening 402, remains spaced-apart from the cervix 404.

FIG. 4B illustrates the CMS 300 after transition to the expanded condition. As shown in FIG. 4B, the axial compression of the CMS 300 increases the outer diameter 322 of the CMS 300 and decreases the overall length 320 of the CMS 300 in the expanded condition as compared to the collapsed condition. With the CMS 300 in the expanded condition, the vaginal walls 406 are further separated, which enables for the control and adjustment of the position of the cervix 404. In one example, the CMS 300 may be adjusted in the direction(s) illustrated by the arrows 412 and 414 (as well as other suitable directions) to reposition or otherwise create easier access to the cervix 404. The friction-enhancing material 330, together with the force applied by the expanded CMS 300, facilitates this adjustment of the cervix 404 via the CMS 300.

In some embodiments, with the CMS 300 in the expanded condition and having repositioned the cervix 404 as desired, at least one instrument “I” may be inserted through the channel 332 of CMS 300 (see FIGS. 3A′ and 3B′). The instrument(s) “I” may include an endoscope, a treatment device, an implant, or other suitable instrument(s) “I” configured to facilitate performing one or more surgical tasks on or within the cervix 404.

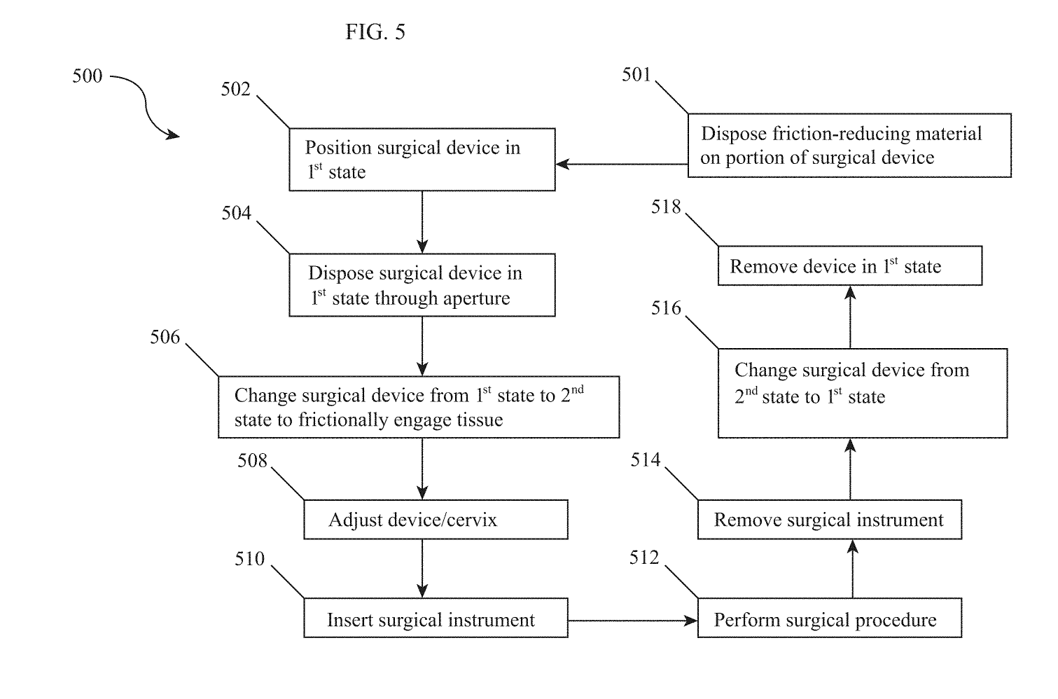

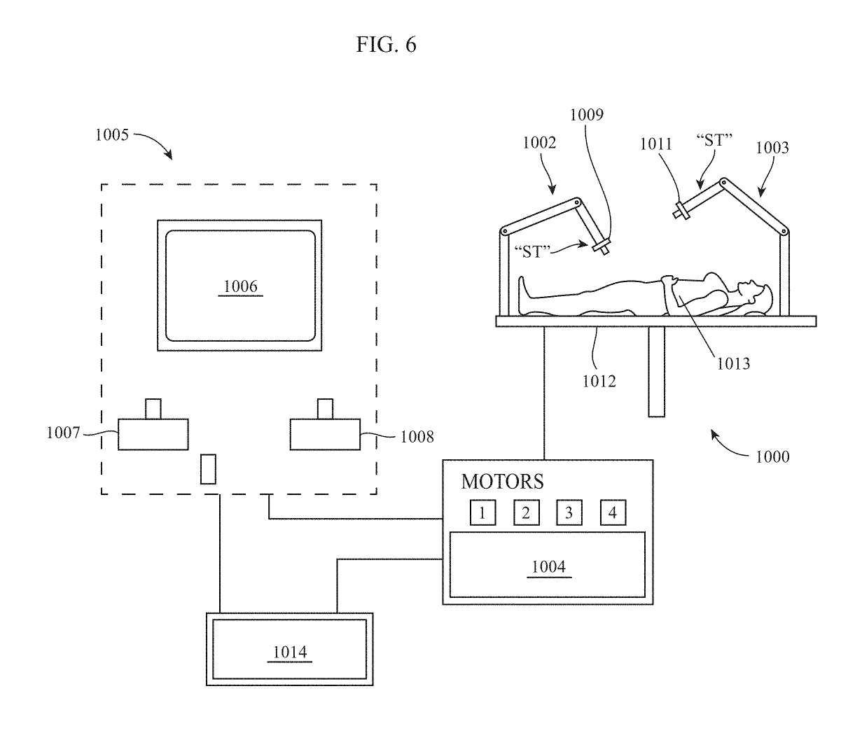

Turning to FIG. 5, a method 500 of surgery according to embodiments of the present disclosure is provided. At 501, a friction-enhancing material is disposed on a portion of a surgical device, e.g., forceps 100 (FIG. 1), speculum 200 (FIG. 2), CMS 300 (FIGS. 3A-3B′), or other suitable surgical device. The friction-enhancing material may be applied by the OEM and, thus, present on the surgical device when it is received from the OEM. In such configurations, 501 has already been performed and need not (but may) be repeated by a member of the medical team. Alternatively or additionally, at 501, the friction-enhancing material may be applied and/or enhanced by a member of the medical team.

At 502, the surgical device, including the friction-enhancing material, is disposed in a first state, e.g., corresponding to the closed position of forceps 100 (FIG. 1) and speculum 200 (FIG. 2) or the collapsed condition of CMS 300 (FIG. 3A). At 504, the surgical device is advanced through a body aperture, e.g., the vagina, while in the first state, and is moved into position, e.g., within the vagina and remaining spaced-apart from the cervix. Thereafter, at 506, the surgical device is changed from the first state to a second state, e.g., corresponding to the open position of forceps 100 (FIG. 1) and speculum 200 (FIG. 2) or the expanded condition of CMS 300 (FIG. 3B), to frictionally engage tissue, e.g., the vaginal walls. The surgical device may then be adjusted at 508 to adjust and stabilize the cervix as desired, facilitated by the friction-enhancing material, as detailed above.

With the cervix adjusted to a desired position and stabilized thereat, a surgical instrument may be inserted, at 510, through the surgical device while it is in the second state and advanced to or through the cervix. At 512, the surgical instrument is utilized to perform a surgical task on or within the cervix, e.g., visualizing the cervix, applying an implant, treating tissue, removing tissue, etc. Finally, the surgical instrument is removed at 514. 510, 512, and 514 may be repeated in any suitable order and/or in any number of repetitions to enable use of different instruments, and maybe repeated serially or in concomitance.

At 516, the surgical device is changed from the second state back to the first state, and the surgical device is removed from the body aperture at 518.

Referring to FIG. 4, a robotic surgical system configured for use in accordance with the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST.” One of the surgical tools “ST” may include a distal end portion similar to that of forceps 100 (FIG. 1) or speculum 200 (FIG. 2), or may include a CMS 300 (FIGS. 3A-3B′) operably coupled thereto. The surgical tool “ST”, more specifically, may be operably coupled to drive bars, drive cables, pulleys, rotational motors, translationary motors, etc. so as to provide similar functionality as manual actuators, e.g., handles 411 (FIGS. 4A and 4B). The other surgical tool “ST” may include an instrument “I” (FIG. 4B) such as an endoscope or other visualization device, a surgical instrument for performing a surgical task on or within the cervix, an implant, etc.

Robot arms 1002, 1003 may be driven by electric drives, e.g., motors, connected to control device 1004. Control device 1004, e.g., a computer, may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

What is claimed is:

1. A method, comprising:
advancing a surgical device into a vaginal opening of a subject to a position proximal of a cervix of the subject;
transitioning the surgical device from a first state to a second state such that an outer surface of the surgical device applies force to vaginal walls of the subject in a radially outward direction relative to a longitudinal axis of the surgical device to frictionally engage the vaginal walls of the subject; and
moving the surgical device in a caudal direction to manipulate the cervix of the subject.

2. The method of claim 1, wherein the surgical device is a speculum including a handle and first and second arms coupled to the handle, and wherein transitioning the surgical device from the first state to the second state includes moving the first and second arms from a closed position to an open position.

3. The method of claim 1, wherein the surgical device is a forceps including a handle and first and second arms coupled to the handle, and wherein transitioning the surgical device from the first state to the second state includes moving the first and second arms from a closed position to an open position.

4. The method of claim 1, wherein the surgical device is a collapsible mesh structure (CMS), and wherein transitioning the surgical device from the first state to the second state includes expanding the CMS from a radially collapsed condition to a radially expanded condition.

5. The method of claim 4, wherein expanding the CMS from the radially collapsed condition to the radially expanded condition includes applying compressive force along a longitudinal axis of the CMS.

6. The method of claim 4, wherein, in the radially collapsed condition, the CMS defines a first length, and wherein, in the radially expanded condition, the CMS defines a second length less than the first length.

7. The method of claim 1, wherein the surgical device includes a friction enhancing material disposed on the outer surface thereof, the friction enhancing material increasing frictional engagement with the vaginal walls of the subject upon movement of the surgical device in the caudal direction.

8. The method of claim 1, further comprising inserting a surgical instrument through the surgical device and into the cervix of the subject.

9. The method of claim 8, further comprising performing at least one surgical task on or within the cervix of the subject with the surgical instrument.

10. The method of claim 9, further comprising:
removing the surgical instrument;
transitioning the surgical device from the second state back to the first state; and
removing the surgical device from the vaginal opening of the subject.

11. The method of claim 1, further comprising, prior to advancing a surgical device into the vaginal opening of the subject, applying a friction-enhancing material to the outer surface of the surgical device.

12. A system, comprising:
a surgical device defining an outer surface and a longitudinal axis and configured for insertion into a vaginal opening of a subject, the surgical device transitionable between a first state, wherein the surgical device defines a first radial dimension, and a second state, wherein the surgical device defines a second radial dimension greater than the first radial dimension, wherein in the second state, at least a portion of the outer surface of the surgical device is configured to frictionally engage vaginal walls of the subject; and
a friction-enhancing material disposed on the at least a portion of the outer surface of the surgical device, the friction-enhancing material configured to increase the frictional engagement of the surgical device with the vaginal walls of the subject.

13. The system of claim 12, wherein the surgical device is a speculum including a handle and first and second arms coupled to the handle, and wherein the handle is selectively manipulatable to transition the first and second arms from a closed position, corresponding to the first state, to an open position, corresponding to the second state.

14. The system of claim 12, wherein the surgical device is a forceps including a handle and first and second arms coupled to the handle, and wherein the handle is selectively manipulatable to transition the first and second arms from a closed position, corresponding to the first state, to an open position, corresponding to the second state.

15. The system of claim 12, wherein the surgical device is a collapsible mesh structure (CMS) configured to transition between a radially collapsed condition, corresponding to the first state, and a radially expanded condition, corresponding to the second state.

16. The system of claim 15, wherein, in the radially collapsed condition, the CMS defines a first length and a first diameter, and wherein, in the radially expanded condition, the CMS defines a second length less than the first length and a second diameter greater than the first diameter.

17. The system of claim 15, wherein the CMS includes a plurality of interwoven components.

18. The system of claim 15, wherein the CMS is configured to transition from the radially collapsed condition to the radially expanded condition in response to application of a compressive force thereto.

19. The system according to claim 12, further comprising a surgical instrument configured for insertion through the surgical device when the surgical device is in the second state.

20. The system according to claim 12, wherein the friction-enhancing material is gauze.
